(19) United States
(12) Patent Application Publication
(10) Pub. No.: US 2014/0277146 A1
(43) Pub. Date: Sep. 18, 2014

(54) CROSS-BRACED BILATERAL SPINAL ROD CONNECTOR

(71) Applicant: BLACKSTONE MEDICAL, INC., Lewisville, TX (US)

(72) Inventors: Nelson Li, Hillsdale, NJ (US); Francesco Larosa, Neptune, NJ (US)

(73) Assignee: BLACKSTONE MEDICAL, INC., Lewisville, TX (US)

(21) Appl. No.: 13/836,504

(22) Filed: Mar. 15, 2013

(51) Int. Cl. A61B 17/70

(52) U.S. Cl. CPC A61B 17/7052
USPC 606/252; 606/279

(57) ABSTRACT

A spinal rod connector apparatus for use with spinal implantation rods and methods of securing implantation rods using a cross-braced bilateral spinal rod connector apparatus. The spinal rod connector apparatus comprises a central member having two or more extension legs. The extension legs can be of varying lengths and disposed at varying angles with respect to the monolithic central member, depending upon physiological sizing requirements. Attached to each extension leg is a connecting member capable of connecting to a bilateral spinal rod construct.

### BACKGROUND

The present disclosure relates to a support rod connector apparatus for use with, inter alia, spinal implantation rods and related methods of securing implantation rods using a cross-braced bilateral spinal rod connector apparatus. More particularly, this disclosure relates to a novel, cross-braced bilateral support rod connector that enhances the rigidity of bilateral spinal rods anchored to the spine.

The bones and connective tissue of an adult human spinal column consists of more than 20 discrete bones coupled sequentially to one another by a tri-joint complex. The complex consists of an anterior disc and two posterior facet joints. The anterior discs of adjacent bones are cushioned by cartilage spacers referred to as intervertebral discs. The over 20 bones of the spinal column are anatomically categorized as one of four classifications: cervical, thoracic, lumbar, or sacral. The cervical portion of the spine which comprises the top of the spine up to the base of the skull, includes the first 7 vertebrae. The intermediate 12 bones are thoracic vertebrae, and connect to the lower spine comprising the 5 lumbar vertebrae. The base of the spine are sacral bones, including the coccyx.

The spinal column of bones is highly complex in that it includes over 20 bones coupled to one another, housing and protecting critical elements of the nervous system having innumerable peripheral nerves and circulatory bodies in close proximity. Despite its complexity, the spine is a highly flexible structure, capable of a high degree of curvature and twist in nearly every direction.

Genetic or developmental irregularities, trauma, chronic stress, tumors and disease, however, can result in spinal pathologies which either limit this range of motion or threaten the critical elements of the nervous system housed within the spinal column. A variety of systems have been disclosed in the art which achieve immobilization by implanting artificial assemblies in or on the spinal column. These assemblies may be classified as anterior, posterior or lateral implants. Lateral and anterior assemblies are coupled to the anterior portion of the spine which is in the sequence of vertebral bodies. Posterior implants generally comprise pairs of rods (“bilateral spinal support rods”), which are aligned along the axis which the bones are to be disposed, and which are then attached to the spinal column by either hooks which couple to the lamina or attach to the transverse processes, or by screws which are inserted through pedicles.

In order to provide enhanced torsional rigidity to bilateral spinal support rods, these implants generally include cross-connecting devices which couple the rods together, transverse to the axis of the implants. These cross-connecting devices may couple directly to the rods themselves (“rod-to-rod connectors”) or may be attached to pedicle screws (“head-to-head connectors”).

Exemplary prior art references include U.S. Pat. Nos. 5,005,562, 5,261,907, 5,334,203, 5,443,465, 5,601,552, 5,651,789, 5,667,507, 5,688,272, 5,716,355, and 5,947,966.

It is desirable to provide cross-connecting devices to increase the rigidity of bilateral spinal rod construct by providing support axially and longitudinally to support the rod construct.

### BRIEF SUMMARY

The present invention relates to a spinal rod connector apparatus and methods for implementing same. In particular, the connector apparatus comprises a central member with extension legs, wherein the extension legs are capable of connecting to cylindrical rods, such as a bilateral spinal support rods.

In one embodiment, the spinal rod connector apparatus comprises a monolithic central member with two or more extension legs. The extension legs can be of varying lengths and disposed at varying angles with respect to the monolithic central member, depending upon physiological sizing requirements. Attached to each extension leg is a connecting member capable of connecting to a bilateral spinal rod construct. Each connecting member is translatable along and rotatable about the axis of the corresponding extension leg.

In another embodiment, the connecting member is capable of connecting directly to spinal rod on a bilateral spinal rod construct.

In yet another embodiment, the connecting member is capable of connecting directly to a pedicle screw on a bilateral spinal rod construct.

In another embodiment, the monolithic central member is capable of manipulation for additional angle changes through instrumentation in a manner similar to bending of a spinal rod for additional curvature.

In another embodiment, each connecting member comprises a connecting member comprising a slotted end, wherein the slotted end has a slot sized to receive a spinal rod of a bilateral spinal rod construct, the slot being freely rotatable about the axis of the associated extension leg.

In still another embodiment, the spinal rod connector apparatus comprises an angle-adjustable central member with four extension legs. The angles between each of the four extension legs can be adjusted, depending upon physiological sizing requirements. Moreover, the legs can be of varying lengths. Attached to each of the extension legs is a connecting member capable of connecting to a bilateral spinal rod construct. Each connecting member is translatable along and rotatable about the axis of the associated extension leg.

In yet another embodiment, a method for attaching the cross-braced bilateral rod connector apparatus comprises sequentially sliding the connecting members from a lateral approach and engaging a fastening device to secure same to the bilateral rod connector apparatus.

The benefits of this device over conventional head-to-head connectors is the bracing provided at the unsupported span between the support rods to distribute loading to adjacent joints, contrary to conventional head-to-head connectors which only provide support to screws connected to the same vertebral body. As a result, conventional head-to-head connectors only create a stiffer vertebral body by maintaining a constant spacing between the pedicle screws. This distribution of load across adjacent joints alleviates stress shielding and overloading of adjacent joints.

Compared to older rod-to-rod connectors, the cross-braced version provides the same aforementioned stress distribution benefits and alleviates stress concentration/overloading of the rod itself due to the single load path inherent in the older rod-to-rod connector design.

Variations and modifications from the above-described embodiments exist.

### BRIEF DESCRIPTION OF THE DRAWINGS

FIG. 1 is a perspective view of an embodiment of the support rod connector apparatus of the present invention.

FIG. 2 is a perspective view of an embodiment of the support rod connector apparatus as used in a bilateral spinal rod construct.

FIG. 3 is another perspective view of an embodiment of the bilateral support rod connector apparatus as used in a bilateral spinal rod construct.

FIG. 4 is a top plan view of an embodiment of the support rod connector apparatus as used in a bilateral spinal rod construct.

FIG. 5 is another perspective view of an embodiment of the support rod connector apparatus as used in a bilateral spinal rod construct.

FIG. 6 is another perspective view of an embodiment of the support rod connector apparatus as used in a bilateral spinal rod construct.

FIG. 7 is another perspective view of an embodiment of the support rod connector apparatus as used in a bilateral spinal rod construct.

FIG. 8 is a perspective view of an embodiment of the support rod connector apparatus illustrating the translational and rotational ability of the connecting members.

FIG. 9 is a detail perspective view of the extension leg and connecting member of the support rod connector apparatus as connected to a bilateral spinal rod construct.

FIG. 10 is a cross sectional view of the inner mechanism of the connecting member in an embodiment, which is used for connecting the extension leg to the bilateral spinal rod construct.

FIG. 11 is a cross sectional view of the inner mechanism of the connecting member engaged with the extension leg in an embodiment, which is used for connecting the extension leg to the bilateral spinal rod construct.

FIG. 12 is a perspective view of an embodiment of the support rod connector apparatus of the present invention.

FIG. 13 is a perspective view of an embodiment of the support rod connector apparatus of the present invention.

FIG. 14 is a perspective view of a central pivot locking mechanism in an embodiment of the support rod connector apparatus of the present invention.

### DETAILED DESCRIPTION

FIG. 1 illustrates an embodiment of a spinal rod connector apparatus (10). Here, a monolithic central member (1) has four extension legs (2). By forming the central member (1) and the extension legs (2) from a monolithic biocompatible material, such as surgical stainless steel or titanium, the strength and rigidity of the connector apparatus can be increased. In addition, by eliminating a mechanical interface between the central member (1) and the extension legs (2), the potential for weakening, metal fatigue, or loosening of the interface between the central member (1) and the extension legs (2) can be reduced. This improves the effective lifetime of the device (10).

Attached to each extension leg (2) is a connecting member (3) comprising a set-screw (4) and a slot (5). Each connecting member (3) is capable of translating along the longitudinal axis of the associated extension leg (2). Further, each connecting member (3) is capable of complete rotation about the axis of the corresponding extension leg (2).

FIGS. 2-7 illustrate embodiments of the connector apparatus (10) as used in a bilateral spinal rod construct. Here, each connecting member (3) is provided with a slotted opening (5) capable of receiving a spinal rod (6). In FIGS. 2-7, the connecting members (3) are engaged with the spinal rods (6), allowing the connector apparatus (10) to span the unsupported distance between the spinal rods in a cross-braced fashion. Once aligned in the desired position, each connecting members (3) can be semi-permanently locked in said position by tightening of the associated set-screw (4). As set screw (4) is tightened, it pushes against the spinal rod (6), locking it in place while simultaneously fixing the axial and longitudinal position of the connecting member (3). By utilizing one set screw (4) per connecting member (3), complexity and time required for installation of this device is reduced. The head of the set screw (4) has a recess dimensioned and sized to receive a drill bit or screwdriver so that the set screw (4) can be tightened or loosened to the locked or unlocked position, as needed by the surgeon.

FIG. 8 illustrates the capability of the connecting members (3) to translate along the longitudinal axis of the extension legs (2) and rotate about the extension legs.

FIG. 9 illustrates a detailed view of an embodiment of a connecting member (3) engaging with a support rod (6). The support rod (6) is inserted through the slot (5) and semi-permanently engaged in position by tightening of the set screw (4).

FIGS. 10 and 11 illustrate a cross-sectional view of the inner mechanism of an embodiment of the connecting member (3) used for the extension leg (2) and support rod (6) attachment. The connecting member (3) includes a connection aperture (105) through which an extension leg (not shown) can be received. A set screw (4) is depicted at the superior end (110) of the connecting member (3). The set screw (4) also interfaces with a side of the connection aperture (105) such that the set screw (4) can be driven against an extension leg (2) by applying a torque to the set screw (4). Also depicted in FIG. 10 is a compression saddle (7) that is located on an opposite side of the connection aperture (105) from the set screw (4). The compression saddle (7) interfaces with the extension leg (2) such that when a compressive force is applied to the extension leg by the set screw (4), the compression saddle (7) is driven downwards towards the slot (5). If a spinal rod (6) is located within the slot (5) (e.g., when the connecting member (3) is being attached to the spinal rod (6), the downward force of the compression saddle (7) will lock the connecting member (3) to the spinal rod (6). Thus, by driving the set screw (4) with an appropriate torque, the connecting member (3) will lock the position of the extension leg (2) with respect to the spinal rod (6) into a rigid assembly. Prior to tightening, the connecting member (3) is free to rotate about the extension leg (2) and slide along the spinal rod (6). This provides a degree of freedom for the surgeon to adjust the device to suit the specific needs of the patient. Once suitable adjustments are made, the device is tightened as described above.

FIGS. 12-13 illustrate embodiments of a support rod connector apparatus with a central pivot locking mechanism. Here, the central member (1) comprises a central pivot locking mechanism (8). Further, the central member has four extension legs (2). Attached to each extension leg (2) is a connecting member (3) comprising a set-screw (4) and a slot (5). Each connecting member (3) is capable of translating along the longitudinal axis of associated extension leg (2). Further, each connecting member (3) is capable of complete rotation about the axis of the associated extension leg (2). FIG. 12 illustrates that the angle (α) between the extension legs (2) is relatively small, whereas FIG. 13 illustrates a relatively larger angle (β) between the extension legs (2).

FIG. 14 illustrates a detail view of the central member (1), wherein the central member (1) comprises a central pivot locking mechanism (8). The central pivot locking mechanism (8) provides for ease of use when surgeons need to adjust the angles angle between the extension legs (2). The central pivot locking mechanism includes a releasable locking mechanism, such as a set screw (115). When the releasable locking mechanism (8) is released (e.g., when the set screw (115) is loosened), the angle between the extensions legs (2) can be adjusted within a range of angles, depending up the preference of the surgeon, the anatomy of the patient, or the location of existing pedicle screws along the bilateral spinal rods. According to another embodiment, each of the extension legs can be bent, turned, or otherwise adjusted to accommodate the curvature of the spine, and thus the curvature of the corresponding bilateral spinal rods.

While various embodiments in accordance with the disclosed principles have been described above, it should be understood that they have been presented by way of example only, and are not limiting. Thus, the breadth and scope of the invention(s) should not be limited by any of the above-described exemplary embodiments, but should be defined only in accordance with the claims and their equivalents issuing from this disclosure. Furthermore, the above advantages and features are provided in described embodiments, but shall not limit the application of such issued claims to processes and structures accomplishing any or all of the above advantages.

Additionally, the section headings herein are provided for consistency with the suggestions under 37 C.F.R. 1.77 or otherwise to provide organizational cues. These headings shall not limit or characterize the invention(s) set out in any claims that may issue from this disclosure. Specifically, a description of a technology in the “Background” is not to be construed as an admission that technology is prior art to any invention(s) in this disclosure. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings herein.

We claim:

1. A bilateral spinal rod connector apparatus, comprising:
a central member comprising two or more extension legs;
the extension legs comprising one or more connecting members capable of connecting to a bilateral spinal rod construct;
wherein, each connecting member is translatable along and rotatable about a corresponding extension leg.

2. The apparatus of claim 1, wherein the central member comprises at least four extension legs.

3. The apparatus of claim 1, wherein the angle between the extension legs is adjustable.

4. The apparatus of claim 3, wherein the central member comprises a central pivot locking mechanism, whereby the angle between the extension legs can be adjusted and locked.

5. The apparatus of claim 1, wherein the connecting members are capable of connecting directly to a bilateral spinal rod construct.

6. The apparatus of claim 1, wherein the connecting members are capable of connecting directly to pedicle screws of a bilateral spinal rod construct.

7. The apparatus of claim 1, wherein the central member and the extension legs are monolithic.

8. The apparatus of claim 1, wherein one or more of the extension legs can be bent to accommodate the anatomy of the patient.

9. The apparatus of claim 1, wherein each connecting member comprises a slotted end, wherein the slotted end comprises a slot sized to receive a spinal rod of the bilateral spinal rod construct, and a connecting aperture through the connecting member operable to receive an extension leg.

10. The apparatus of claim 1, wherein the connecting members are barrel-shaped.

11. The apparatus of claim 9, wherein each connecting member further comprises:
a set screw attached to a superior end of the connecting member and interfacing with a first side of the connecting aperture;
a compression saddle interfacing with an opposite side of the connecting aperture and interfacing with the slot in the slotted end;
wherein, when an extension leg is within the connecting aperture, and a spinal is within the slot, applying a driving force to the set screw in the connecting member will lock the extension leg and the spinal rod to the connecting member.

12. A method of cross-connecting a pair of bi-lateral spinal rods on opposite lateral sides of a vertebral column, the method comprising:
providing a bilateral spinal rod connector apparatus comprising a central member, two or more extension legs, and two or more connecting members corresponding to each of the extension legs, wherein the connector apparatus is operable to connect
translating each of the connecting members along the longitudinal axis of a corresponding extension leg, through a connection aperture in the connecting members;
inserting a spinal rod into a slot in each of the connecting members; and
driving a set screw in each the connecting members to lock the connector apparatus to the corresponding spinal rod.
